(19) United States
(12) Patent Application Publication
(10) Pub. No.: US 2023/0398195 A1
(43) Pub. Date: Dec. 14, 2023

(54) COMPOSITION DERIVED FROM MAMMALIAN UMBILICAL CORD AND WHARTONS JELLY FOR USE IN THERAPEUTIC AND REGENERATIVE APPLICATIONS

(71) Applicant: Equi-Stem Biotechnologies LLC, West Bloomfield, MI (US)

(72) Inventor: Todd Flower, West Bloomfield, MI (US)

(21) Appl. No.: 18/457,488

(22) Filed: Aug. 29, 2023

Related U.S. Application Data
(63) Continuation-in-part of application No. 16/731,911, filed on Dec. 31, 2019.
Application No. 16/731,911 is a continuation-in-part of application No. 15/660,921, filed on Jul. 26, 2017, now abandoned.
(60) Provisional application No. 62/366,623, filed on Jul. 26, 2016.

(51) Int. Cl. A61K 38/52; A61K 38/17; A61K 38/22; A61K 38/18; A61K 31/727; A61K 9/00

(52) U.S. Cl. CPC A61K 38/52; A61K 38/1709; A61K 38/22; A61K 38/18; A61K 31/727; A61K 9/0014; C12Y 503/99004

(57) ABSTRACT

A solution for topical application to the skin for therapeutic and regenerative applications comprises about 0.05% by weight of annexin-1, about 0.76% by weight of galectin-1, about 0.23% by weight of protein S-100, about 0.003% by weight of timp-1, about 0.01% by weight of timp-2, about 0.01% by weight of ECM1, about 0.2% by weight of adiponectin, about 0.002% by weight of nephroblastoma overexpressed protein, about 0.0003% by weight of prostacyclin synthase, about 0.001% by weight of C-X-X motif chemokine, about 2% by weight of heparan sulphate, and about 0.01% by weight of apolipoprotein D.

### CROSS-REFERENCE TO RELATED APPLICATIONS

This application is a continuation-in-part of U.S. Non-Provisional patent application Ser. No. 16/731,911, filed Dec. 31, 2019, which is a continuation-in-part of U.S. Non-Provisional patent application Ser. No. 15/660,921, filed Jul. 26, 2017, which claims the benefit of U.S. Provisional Application No. 62/366,623, filed Jul. 26, 2016. The subject matter of patent application Ser. No. 16/731,911, 15/660,921, 62/366,623 is incorporated herein by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The disclosed subject matter relates generally to the field of medicine and wellness, and, more specifically, relates to compositions with therapeutic and regenerative properties useful for the treatment of inflammation, wound healing, muscle healing/regeneration, articular pain, neurologic disorders and arthritis.

### BACKGROUND

Umbilical cord tissue and Wharton's jelly is a tissue that surrounds the umbilical cord vessels. This tissue contains high amounts of extracellular matrix (ECM) components, mainly hyaluronic acid, collagen, and several sulphated proteoglycans and naïve cells that are referred to a Mesenchymal Stem Cells (MSCs). A large number of growth factors and cytokines have been found to associate with extracellular matrix proteins. These growth factors and cytokines can control cell proliferation, inflammation, wound healing and remodeling, burns, anti-fibrotic (anti-scarring activity) and the synthesis and remodeling of the extracellular matrix. The claimed subject matter describes the extraction of these factors from umbilical cord tissue and their human and vet applications to treat a variety of conditions in both humans and animals.

Wharton's jelly, named after the person who first described it in his publication Adenographia, “The Description of the Glands of the Entire Body” published in 1656, is a gelatinous tissue which surrounds the umbilical cord vessels within the umbilical cord that contains myo-fibroblast-like stromal cells. See FIG. 1 for a cross sectional view of an umbilical cord. The extracellular matrix of umbilical cord is the mammalian tissue with one of the highest contents of Hyaluronic Acid (HA).

The umbilical cord forms the connection between the placenta and the fetus. It contains one vein and two (2) arteries surrounded by a myxomatous substance called Wharton's jelly, consisting of stem cells, high amounts of extracellular matrix components mainly collagen, hyaluronate and several sulphated proteoglycans. The large amount of hyaluronate make this tissue highly hydrated, whereas the abundant content of collagen makes it resistant to extension, bending, twisting and compression evoked by fetal movements and uterine contractions. Furthermore, the extracellular matrix of Wharton's jelly is an abundant reservoir of numerous cytokines, peptides and peptide growth.

The main components of umbilical cord tissue and Wharton's jelly are proteoglycans, macromolecules built of protein cores covalently attached to sulphated glycosaminoglycans. Proteoglycans perform numerous functions including affecting the mechanical properties of tissues, regulate collagen matrix organization, participate in cell-cell and cell-extracellular matrix interactions, bind growth factors, enzymes, viruses, etc.

The above components serve as extracellular matrix components which are secreted molecules that constitute the cell microenvironment, composed of a dynamic and complex array of glycoproteins, collagens, glycosaminoglycans and proteoglycans. The extracellular matrix provides the bulk, shape and strength of many tissues. However, the extracellular matrix provides much more than just mechanical and structural support. ECM molecules can be flexible and extendable and mechanical tension can expose cryptic sites, which could further interact with growth factors, signaling molecules or their receptors.

Extracellular matrix (ECM) proteins play crucial and complex roles during cell surface receptor signaling. The ECM serves as a reservoir for growth factors and signaling molecules. ECM-bound growth factors are released and bind to their specific receptors. Many ECM proteins have binding sites for both cell adhesion and growth factors, allowing local concentration of the growth factors near to their cell surface receptors and cell adhesion sites.

Some of the growth factors and signaling molecules found in Wharton's jelly are involved in a wide variety of different cytokines and growth factors including involved in anti-cancer, wound healing, neuroprotection, liver protection, anti-inflammatory, etc. These specific factors include but are not limited to the factors defined below:

EGF—Epidermal Growth Factor: a mitogenic polypeptide produced by many cell types and made in large amounts by some tumors. It promotes growth and differentiation, is essential in embryogenesis, and is also important in wound healing. It has been found to be part of a family of compounds that includes also transforming growth factor.

PDGF—Platelet Derived Growth Factor: one of the numerous growth factors, or proteins that regulate cell growth and division. In particular, it plays a significant role in blood vessel formation (angiogenesis), the growth of blood vessels from already-existing blood vessel tissue.

aFGF—Acidic Fibroblast Growth Factor and bFGF—Basic Fibroblast Growth Factor: In normal tissue, basic fibroblast growth factor is present in basement membranes and in the subendothelial extracellular matrix of blood vessels. It stays membrane bound as long as there is no signal peptide. It has been hypothesized that, during both wound healing of normal tissues and tumor development, the action of heparan sulfate-degrading enzymes activates bFGF, thus mediating the formation of new blood vessels, a process known as angiogenesis. In addition, it is synthesized and secreted by human adipocytes and the concentration of bFGF correlates with the BMI in blood samples. In this study, bFGF was also shown to act on preosteoblasts—in the form of an increased proliferation—after binding to fibroblast growth factor receptor 1 and activating phosphoinositide 3-kinase. bFGF has been shown in preliminary animal studies to protect the heart from injury associated with a heart attack, reducing tissue death and promoting improved function after reperfusion.

IGF-1—Insulin Like Growth Factor 1: The primary action is mediated by binding to its specific receptor, the insulin-like growth factor 1 receptor (IGF1R), which is present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death.

TGF-β—Transforming Growth Factor Beta: Involved in many cellular processes in both the adult organism and the developing embryo including cell growth, cell differentiation, apoptosis, cellular homeostasis and other cellular functions.

BDNF—Brain Derived Neurotropic Factor: BDNF acts on certain neurons of the central nervous system and the peripheral nervous system, helping to support the survival of existing neurons, and encourage the growth and differentiation of new neurons and synapses. In the brain, it is active in the hippocampus, cortex, and basal forebrain—areas vital to learning, memory, and higher thinking.

GDNF—Glial Derived Neurotrophic Factor GDNF: A protein that, in humans, is encoded by the GDNF gene. GDNF is a small protein that potently promotes the survival of many types of neurons. It signals through GFRα receptors, particularly GFRα1.

aG-CSF—Granulocyte Colony Stimulating Factor (G-CSF or GCSF), also known as colony-stimulating factor 3 (CSF 3), is a glycoprotein that stimulates the bone marrow to produce granulocytes and stem cells and release them into the bloodstream. Functionally, it is a cytokine and hormone, a type of colony-stimulating factor, and is produced by a number of different tissues. The pharmaceutical analogs of naturally occurring G-CSF are called filgrastim and lenograstim.

SDF-1—Stromal Cell Derived Factor 1 CXCL12: Plays an important role in angiogenesis by recruiting endothelial progenitor cells (EPCs) from the bone marrow through a CXCR4 dependent mechanism.

PDGF-AA—Platelet-Derived Growth Factor AA: PDGF-AA is one of the numerous growth factors, or proteins that regulate cell growth and division. In particular, it plays a significant role in blood vessel formation (angiogenesis), the growth of blood vessels from already-existing blood vessel tissue.

Angiopoietin-2: Angiopoietin is part of a family of vascular growth factors that play a role in embryonic and postnatal angiogenesis. Angiopoietin signaling most directly corresponds with angiogenesis, the process by which new arteries and veins form from preexisting blood vessels.

VEGF—Vascular Endothelial Growth Factor: Vascular endothelial growth factor (VEGF), originally known as vascular permeability factor (VPF), is a signal protein produced by cells that stimulates vasculogenesis and angiogenesis. It is part of the system that restores the oxygen supply to tissues when blood circulation is inadequate such as in hypoxic conditions.

CXCL-16—Chemokine Ligand 16 (CXCL) 16: One of the ELR—CXC chemokines, acts as a mediator of innate immunity by attracting CXC chemokine receptor (CXCR) 6-expressing cells, such as activated T cells and NKT cells.

NAP-2—Neutrophil-Activating Protein-2: Chemokine (C-X-C motif) ligand (CXCL7) is a small cytokine belonging to the CXC chemokine family. It is a protein that is released in large amounts from platelets following their activation. It stimulates various processes including mitogenesis, synthesis of extracellular matrix, glucose metabolism and synthesis of plasminogen activator.

(GITR) Glucocorticoid-induced Tumor Necrosis Factor Receptor: GITR is currently considered to be a co-stimulatory immune checkpoint molecule.

FGF-20 Fibroblast Growth Factor 20: FGF family members possess broad mitogenic and cell survival activities, and are involved in a variety of biological processes, including embryonic development cell growth, morphogenesis, tissue repair, tumor growth and invasion. This gene was shown to be expressed in normal brain, particularly the cerebellum. The rat homolog is preferentially expressed in the brain and able to enhance the survival of midbrain dopaminergic neurons in vitro.

IL-10—Interleukin-10: Interleukin 10 (IL-10), also known as human cytokine synthesis inhibitory factor (CSIF), is an anti-inflammatory cytokine. In humans, interleukin 10 is encoded by the IL10 gene.

IL-12—Interleukin-12: Interleukin 12 (IL-12) is an interleukin that is naturally produced by dendritic cells, macrophages, neutrophils, and human B-lymphoblastoid cells (NC-37) in response to antigenic stimulation.

IL-13—Interleukin-13: IL-13 has effects on immune cells that are similar to those of the closely related cytokine IL-4. However, IL-13 is suspected to be a more central mediator of the physiologic changes induced by allergic inflammation in many tissues. Although IL-13 is associated primarily with the induction of airway disease, it also has anti-inflammatory properties. IL-13 induces a class of protein-degrading enzymes, known as matrix metalloproteinases (MMPs), in the airways. These enzymes are required to induce egression of effete parenchymal inflammatory cells into the airway lumen where they are then cleared.

IL-15—Interleukin-15: Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

IL-17A—Interleukin 17A: Signaling from IL-17 recruits monocytes and neutrophils to the site of inflammation in response to invasion by pathogens, similar to Interferon gamma. In promoting inflammation, IL-17 has been demonstrated to act synergistically with tumor necrosis factor and interleukin-1 This activity can also be redirected towards the host and result in various autoimmune disorders that involve chronic inflammation, such as the skin disorder psoriasis.

IL-1RA Interleukin-1 Receptor Agonist: IL-1RA is a member of the interleukin 1 cytokine family. IL1Ra is secreted by various types of cells including immune cells, epithelial cells, and adipocytes, and is a natural inhibitor of the pro-inflammatory effect of IL1β. This protein inhibits the activities of interleukin 1, alpha (ILIA) and interleukin 1, beta (IL1B), and modulates a variety of interleukin 1 related immune and inflammatory responses. This gene and five other closely related cytokine genes form a gene cluster spanning approximately 400 kb on chromosome 2. Four alternatively spliced transcript variants encoding distinct isoforms have been reported.

IL-9—Interleukin-9: This cytokine stimulates cell proliferation and prevents apoptosis. It functions through the interleukin-9 receptor (IL9R), which activates different signal transducer and activator (STAT) proteins and thus connects this cytokine to various biological processes.

IL-2—Interleukin-2: Interleukin-2 (IL-2) is an interleukin, a type of cytokine signaling molecule in the immune system. It is a protein that regulates the activities of white blood cells (leukocytes, often lymphocytes) that are responsible for immunity.

IL-3—Interleukin-3: Interleukin-3 (IL3) is a cytokine that regulates blood-cell production by controlling the production, differentiation and function of granulocytes and macrophages.

IL-4—Interleukin-4: The interleukin 4 (IL4) is a cytokine that induces differentiation of naive helper T cells (Th0 cells) to Th2 cells. Upon activation by IL-4, Th2 cells subsequently produce additional IL-4 in a positive feedback loop.

IL-5—Interleukin-5: Interleukin-5 is produced in lymphocytes, mast cells, eosinophils, and airway smooth muscle and epithelial cells, and is primarily responsible for the maturation and release of eosinophils in the bone marrow.

IL-6—Interleukin-6: Interleukin 6 (IL-6) is an interleukin that acts as both a pro-inflammatory cytokine and an anti-inflammatory myokine.

IL-7—Interleukin-7: Interleukin 7 (IL-7) is a protein that in humans is encoded by the IL7 gene. IL-7 is a hematopoietic growth factor secreted by stromal cells in the bone marrow and thymus. It is also produced by keratinocytes, dendritic cells, hepatocytes, neurons, and epithelial cells but is not produced by normal lymphocytes

IL-8—Interleukin-8: Interleukin-8, also known as neutrophil chemotactic factor, has two primary functions. It induces chemotaxis in target cells, primarily neutrophils but also other granulocytes, causing them to migrate toward the site of infection. IL-8 also induces phagocytosis once they have arrived. IL-8 is also known to be a potent promoter of angiogenesis. In target cells, IL-8 induces a series of physiological responses required for migration and phagocytosis, such as increases in intracellular Ca2+, exocytosis (e.g., histamine release), and the respiratory burst.

MCP-1—Monocyte Chemotactic Protein 1: The chemokine (C-C motif) ligand 2 (CCL2) is also referred to as monocyte chemoattractant protein 1 (MCP1) and small inducible cytokine A2. CCL2 is a small cytokine that belongs to the CC chemokine family. CCL2 recruits monocytes, memory T cells, and dendritic cells to the sites of inflammation produced by either tissue injury or infection.

With respect to ratios, Wharton's jelly contains a small number of cells compared to the amount of extracellular matrix components. Based on this fact, it can be concluded that the cells present in Wharton's jelly are strongly stimulated to produce large amounts of collagen, hyaluronic acid and sulphated proteoglycans.

In addition, the MSCs found in Wharton's jelly have been reported to secrete a wide variety of different factors (tropism). These secreted trophic factors are able to enhance angiogenesis, synaptogenesis and neurogenesis. These factors also have been shown to activate the PI3K-Akt pathway resulting in the inhibition of apoptosis, increased cell survival and a stimulation of angiogenesis; which is believed to be partly due to the release of angiogenic factors such as IL-6, VEGF, and monocyte chemoattractant protein (MCP)-1. These secreted factors also appear to increase the expression of local neurotransmitters, such as brain-derived neurotrophic factor (BDNF), neurotrophin-3 (NTF-3), which should enhance recovery as well as a variety of different factors that are involved in wound healing. All of these factors are released into the surrounding tissue.

Growth factors and cytokines exert their regulatory roles on various cells by their action on specific receptors. These may be present on the surface of the same cell that produces the growth factors (autocrine action). Alternatively, the growth factors may work on other target cells, which are not themselves the producer cell (paracrine action). In some cases, target cells may also occur in distant parts of the body, giving rise to a type of regulation analogous to the mode of action of polypeptide hormones (endocrine regulation).

Consequently, a further need exists to overcome the problems with the prior art as discussed above, and particularly for a more efficient and expeditious way of promoting healing and other therapeutic and regenerative effects in humans and other animals.

### SUMMARY

This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The claimed solution is a complex formulation intended for topical application to the skin. It includes a variety of proteins, each of which have a therapeutic effect based on their known biological functions. The solution for topical application to the skin comprises about 0.05% by weight of annexin-1, about 0.76% by weight of galectin-1, about 0.23% by weight of protein S-100, about 0.003% by weight of timp-1, about 0.01% by weight of timp-2, about 0.01% by weight of ECM1, about 0.2% by weight of adiponectin, about 0.002% by weight of nephroblastoma overexpressed protein, about 0.0003% by weight of prostacyclin synthase, about 0.001% by weight of C-X-X motif chemokine, about 2% by weight of heparan sulphate, and about 0.01% by weight of apolipoprotein D.

To the accomplishment of the above and related objects, the claimed subject matter may be embodied in the form illustrated in the accompanying drawings, attention being called to the fact, however, that the drawings are illustrative only, and that changes may be made in the specific construction illustrated and described within the scope of the appended claims. The foregoing and other features and advantages of the claimed subject matter will be apparent from the following more particular description of the preferred embodiments, as illustrated in the accompanying drawings.

### BRIEF DESCRIPTION OF THE DRAWINGS

The accompanying drawings, which are incorporated in and constitute part of this specification, illustrate embodiments of the claimed subject matter and together with the description, serve to explain the principles of the disclosed embodiments. The embodiments illustrated herein are presently preferred, it being understood, however, that the claimed subject matter is not limited to the precise arrangements and instrumentalities shown, wherein:

FIG. 1 is an illustration of a cross sectional view of an umbilical cord.

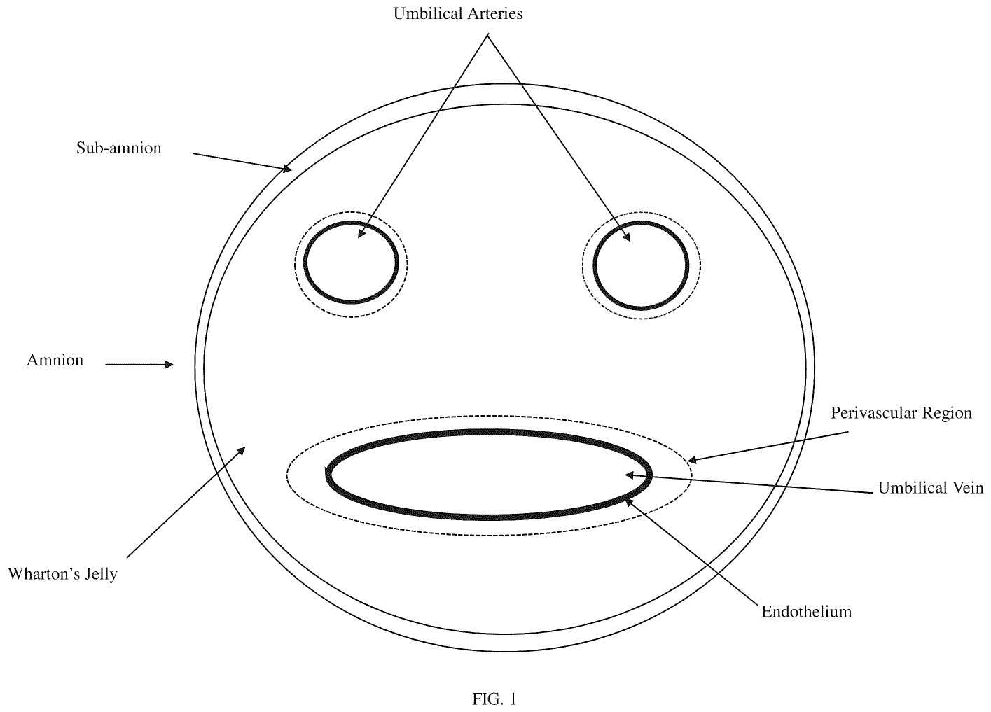

FIGS. 2A, 2B, 2C, and 2D comprise photographs of experimental results of use of the claimed therapeutic agent, according to one embodiment.

FIGS. 3A, 3B, 3C, and 3D comprise photographs of experimental results of the use of the claimed therapeutic agent, including dates and size measurements, according to one embodiment.

FIGS. 4A, 4B, 4C, and 4D comprise photographs of experimental results of the use of the claimed therapeutic agent, including dates and size measurements, according to one embodiment.

FIGS. 5A and 5B comprise photographs of experimental results of the use of the claimed therapeutic agent, including dates and size measurements, according to one embodiment.

FIGS. 6A, 6B, 6C, and 6D comprise photographs of experimental results of the use of the claimed therapeutic agent, including dates, times, locations and descriptions, according to one embodiment.

FIG. 7 is a chart showing inflammation measurements over time, for experimental results, according to one embodiment.

FIG. 8 is a chart showing inflammation measurements over time, for experimental results, according to one embodiment.

FIGS. 9A and 9B comprise photographs of experimental results of the use of the claimed therapeutic agent, according to one embodiment.

FIGS. 10A, 10B, 10C, and 10D comprise photographs of experimental results of the use of the claimed therapeutic agent, according to one embodiment.

FIG. 11 is an illustration of the paracrine effects of stem cells.

FIGS. 12A, 12B, 12C, 12D, 12E, and 12F comprise photographs of an experiment showing MSC growth in RPMI culture media, according to one embodiment.

FIGS. 13A, 13B, 13C, and 13D comprise photographs of an experiment showing MSC growth in RPMI culture media, according to one embodiment, while FIG. 13E shows an MSC growth comparison graph.

### DETAILED DESCRIPTION

The disclosed embodiments are directed to a rapid, inexpensive and easy-to-use therapeutic and regenerative agent that promotes healing, reduces inflammation and treats various afflictions in animals, among other things. The disclosed embodiments improve over the prior art by providing a simple, inexpensive and quick method for producing said therapeutic agent for mass production and transport to individuals and consumers. The disclosed embodiments also improve over the prior art by providing an agent that can be used as cell-culture to promote the growth of desired cells.

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The claimed process produces an acellular product, derived from umbilical cord tissue and Wharton's jelly that contains cytokines, growth factors, peptides, signaling molecules, proteins, RNA, exosomes and anti-inflammatory and regenerative molecules that can be used by itself or combined with a variety of different products including but not limited to; cream-base containing lipophilic agents and can be used to treat anti-inflammatory conditions in mammals (both human and veterinary applications), gel and agarose based products for the delivery of these factors to an external wound in mammals (both human and veterinary applications), can be aerosolized and used via a nebulizer to treat conditions of the lung, can be injected or administered systemically to treat a variety of conditions such as cardiac, neurologic, musculoskeletal, hepatic, and can be lyophilized or freeze-dried and reconstituted for long-term storage and use. The product can also be used as a serum replacement in cell-culture to promote the growth of desired cells.

The disclosed process discloses methods of generating a therapeutic and regenerative product from umbilical cord tissue and Wharton's jelly tissue. In one embodiment, the claimed process provides means of creating a therapeutic and regenerative product useful for the treatment of inflammatory conditions, wounds and degenerative conditions by producing an extract of umbilical cord tissue and Wharton's jelly. Many types of methods of creating a tissue extract may be used and chosen. In one embodiment, a method is selected from a group comprising; 1) sonication; 2) shearing by liquid flow; 3) exploding by pressure; 4) collision forces by impact of beads or paddles; 5) cryogenic grinding; 6) Mortar and Pestle; 7) Glass homogenizer; 8) Blender; 9) Rotor-Stator; 10) Potter-Elvehjem with PTFE Pestle; 11) French Press; 12) Amalgamators for Tubes; 13) High Throughput homogenizers; 14) Combination of the above methods, or any similar methods known in the art.

In some embodiments, the tissue extract is administered directly into the patient (human or animal). It is well known in the art that preparation of the extract before administration may be performed by various means, for example, said extract may be sterile-filtered or in some conditions concentrated or diluted. In one embodiment, the product can be directly administered by injection.

In other embodiments, the tissue extract is used as an active ingredient for the generation of a pharmaceutical formulation. This may comprise administration of the tissue extract therapeutic agent alone, or by way of known pharmaceutical formulations, including tablets, capsules, or elixirs for oral administration, suppositories for rectal administration, sterile solutions or suspensions for parenteral or intramuscular administration, liposomal or encapsulated formulations, formulations wherein the therapeutic agent is alone or conjugated to a delivery agent or vehicle, and the like. The solid form preparations intended to be converted to liquid form may contain, in addition to the active material, flavorings, colorants, stabilizers, buffers, artificial and natural sweeteners, dispersants, thickeners, solubilizing agents, and the like.

It is possible that therapeutic entities of the product will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15.sup.th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the claimed subject matter, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998).

In one embodiment, one or more agents of the claimed process are nanoencapsulated into nanoparticles for delivery. The nanoencapsulation material may be biodegradable or non-degradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic surfactant (BASF). Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen.

These polymers are merely exemplary of the types of nanoencapsulation materials that can be utilized and are not intended to represent all the nanoencapsulation materials within which entrapment is possible. In one embodiment, the therapeutic agent is administered in a topical formulation. Topical formulations are useful in the treatment of conditions associated with dermal diseases and joint disorders/joint pain.

For example, topical administration of the tissue extract may contain aqueous and non-aqueous gels, creams, multiple emulsions, microemulsions, liposomes, ointments, aqueous and non-aqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Topical formulations of the product may include a dermatologically acceptable carrier, e.g., a substance that is capable of delivering the other components of the formulation to the skin with acceptable application or absorption of those components by the skin. The carrier will typically include a solvent to dissolve or disperse the therapeutic agent, and optionally one or more excipients or other vehicle ingredients. Carriers useful in accordance with the topical formulations of the product may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the product are prepared by mixing a compound with a topical carrier.

In other embodiments, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations. A topical formulation may be designed to be left on the skin and not washed shortly after application. Alternatively, the topical formulation may be designed to be rinsed off within a given amount of time after application.

In one aspect, potency of the tissue extract product may be quantified by assessing protein concentration. For quantification of anti-inflammatory and regenerative activity, the term “inflammation” will be understood to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response may be manifested by heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or any condition that has an inflammatory component associated with it, and/or any condition characterized by inflammation as a symptom, including, inter alia, acute, chronic, ulcerative, specific, allergic and necrotic inflammation, and other forms of inflammation. The term thus also includes inflammatory pain.

For the practice of the claimed subject matter supernatants generated by umbilical cord tissue & Wharton's jelly tissue extracts may be administered to the patient in an injection solution, which may be saline, lactated Ringer's solution, mixtures of autologous plasma together with saline, or various concentrations of albumin with saline. Ideally pH of the injection solution is from about 6.4 to about 8.3, optimally 7.4. Excipients may be used to bring the solution to isotonicity such as, 4.5% mannitol or 0.9% sodium chloride, pH buffers, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. Injection can be performed systemically, or more specifically, via routes of administration selected from; a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intranodally; x) by any parenteral route; and y) via inhalation.

The manufacturing of the disclosed product may entail the collection of umbilical cord tissue. The umbilical cord is collected after the new offspring is delivered by clamping the cord proximal to the offspring and then by cutting the cord. The freshly cut umbilical cord is then placed into a container containing buffered saline solution or similar buffered salt solution for transportation to the processing laboratory. The process may proceed by incubating the umbilical cord in a solution containing antibiotics for sterilization at a temperature not exceeding 40 degrees Centigrade.

The umbilical cord is washed in salt buffer about three times and the vasculature can be or may not be removed by micro-dissection and discarded. The dissected tissue is then washed about three more times in a salt buffer to remove any residual blood and homogenized into small fragments (less than 1 mm in size) using a tissue blender (or any other method known in the art), resulting in an aqueous solution. Homogenization is carried out at room temperature for 5 minutes at the highest setting. The umbilical cord may be homogenized in the buffered salt solution so as to produce an aqueous solution comprising umbilical cord tissue proteins, cytokines, growth factors and RNA.

The aqueous solution, including minced tissue, is then re-suspended in a salt buffer and subjected to sonication. This step uses blasts of ultrasonic sound waves to disrupt the cells and tissue to assist in the release of the different cytokines, peptides and peptide growth factors that will be the active components in the treatment of inflammation and regenerative medicine. Sonication occurs via a 300W ultrasonic processing at 20 kHz with a total of 3 second pulse. 2 seconds of sonic pulse followed by 1 second of non-sonic pulse for a duration of 5 minutes. This means there will be 5 minutes of the 2 seconds on, 1 second off pulsing cycle. This results in the liberation of all the proteins necessary for a functional product. The resulting tissue fragments are 1 mm in size after sonication.

After sonication, the aqueous solution (including disrupted tissue and buffer) is transferred into centrifuge tubes and centrifuged at 1,000-15,000 rpm for 10 minutes to pellet all the tissue and cellular debris and to separate the aqueous solution into a soluble component and a non-soluble component. The supernatant containing all the cytokines, peptides, signaling molecules, and peptide growth factors is transferred into a new centrifuge tube and centrifuged again at 1,000-15,000 rpm for 10 minutes to complete the removal and sedimentation of the tissue and cellular debris. In another embodiment, a centrifuge range of 5500-7500 rpm is used. The supernatant is then passed through a 100 μM cell strainer to remove any left-over cellular debris, then it is passed through a 0.22 μM polypropylene syringe filter to sterile filter the supernatant, i.e., the process filtrates and discards the non-soluble component of the aqueous solution from the centrifuge through a 0.2-micron filter. Next, the protein concentration of the soluble component is measured to insure at least about 0.05 microgram of total protein extract up to about 2 micrograms of total protein extract. In one embodiment, the protein concentration of the soluble component is measured to insure 0.05 microgram of total protein extract. In another embodiment, the protein concentration of the soluble component is measured to insure 2 micrograms of total protein extract. The process may further comprise mixing the soluble component with liposomes, resulting in a mix, mixing the mix with cream as a vehicle for application to a mammal, so as to produce a mixed cream, and depositing the mixed cream in a container for transport to users.

The concentration of total protein extract is not recognized as a particular parameter that can be experimented with to increase or optimize the effectiveness of therapeutic and regenerative applications for treating or curing certain maladies. The particular concentration of total protein extract defined above (0.05 microgram and 2.0 micrograms) are significant because they are the result of over 18 months of laboratory and experimental testing of the claimed invention on mammals to find the correct protein extract concentrations, in order to achieve the desired effectiveness. Depending on certain conditions of a mammalian test subject, such as age, gender, height weight and general health, the use of therapeutic and regenerative applications extracted from a mammalian umbilical cord can result in negative side effects on the test subjects, in addition to treating and curing certain maladies.

The total protein extract concentration for a therapeutically effective dose ranges from 0.001 μg/ml to 5 μg/ml and can be varied in order to adjust the effectiveness for specific disease states or treatments (i.e., lung treatment is substantially less concentrated than joint treatment). For example, the therapeutically effective concentration range for administration by inhalation is from 0.001 μg/ml to 0.01 μg/ml; the therapeutically effective concentration range for topical administration is from 0.01 μg/ml to 21 μg/ml; the therapeutically effective concentration range for administration by injection is from 1 μg/ml to 5 μg/ml; and the therapeutically effective concentration range for oral administration is 5 μg/ml. The therapeutically effective concentration ranges recited above are significant because they resulted in decreased negative side effects on the test subjects and increased the effectiveness of the therapeutic and regenerative applications in treating certain maladies—the purpose of the claimed invention.

With regarding to the sonication process, the method described herein includes applying an ultrasonic processer to the aqueous solution to sonicate and disrupt umbilical cord tissue, wherein the ultrasonic processor is applied at a power output of 300 W at a frequency of 20 kHz, wherein sonication occurs in 3 second cycles of 2 seconds on and 1 second off for a total duration of 5 minutes. The sonication profile described above (which include wattage output, frequency and cycle times) is not recognized as a particular parameter that can be experimented with to increase or optimize the effectiveness of producing a therapeutic dose of the claimed solution. The sonication profile described above is significant because it is the result of over 18 months of laboratory and experimental testing of the claimed invention to find the correct sonication profile, in order to achieve the desired therapeutic dose of the claimed solution. Different sonication profiles produce different results in the nature of the resulting solution and its ability to convey a therapeutic dose to the patient.

With regard to the centrifuge process, the method described herein includes applying a centrifuge at 5500-7500 rpm. The centrifuge profile described above is not recognized as a particular parameter that can be experimented with to increase or optimize the effectiveness of producing a therapeutic dose of the claimed solution. The centrifuge profile described above is significant because it is the result of over 18 months of laboratory and experimental testing of the claimed invention to find the correct centrifuge profile, in order to achieve the desired therapeutic dose of the claimed solution.

The claimed solution is a complex formulation intended for topical application to the skin. It includes a variety of proteins, each of which have a therapeutic effect based on their known biological functions. The solution for topical application to the skin, comprises about 0.05% by weight of annexin-1, about 0.76% by weight of galectin-1, about 0.23% by weight of protein S-100, about 0.003% by weight of timp-1, about 0.01% by weight of timp-2, about 0.01% by weight of ECM1, about 0.2% by weight of adiponectin, about 0.002% by weight of nephroblastoma overexpressed protein, about 0.0003% by weight of prostacyclin synthase, about 0.001% by weight of C-X-X motif chemokine, about 2% by weight of heparan sulphate, and about 0.01% by weight of apolipoprotein D. The percentages of each ingredient represent the weight of that ingredient relative to the total weight of the solution. Any uses of the word “about” above may be removed, such that the percentages above are exact numbers. For example, in one embodiment, the claimed solution may comprise 0.05% by weight of annexin-1.

The claimed solution refers to a specialized, intricate blend meant for topical application onto the skin. The constituents of this formulation include an array of proteins, each chosen for their unique biological properties and potential to exert beneficial effects on the skin.

Annexin-1, present at approximately 0.05% by weight, is a protein known for its anti-inflammatory properties. The presence of annexin-1 in the claimed solution might provide anti-inflammatory effects to soothe irritated or inflamed skin conditions, thereby encouraging the process of skin healing and regeneration.

Galectin-1, comprising about 0.76% by weight, is a protein implicated in various biological processes such as cell adhesion, regulation of immune response, and modulation of inflammation. By including galectin-1, the claimed solution could potentially facilitate healing and tissue regeneration.

Protein S-100, incorporated at around 0.23% by weight, represents a family of proteins participating in a myriad of cellular processes, ranging from cell growth to motility. Depending on the specific S-100 protein included, it could contribute to promoting skin healing or modulating immune responses.

TIMP-1 and TIMP-2, at roughly 0.003% and 0.01% by weight respectively, are both tissue inhibitors of metalloproteinases. They are involved in the preservation and remodeling of the extracellular matrix, acting as shields against enzymatic degradation. In the claimed solution, these proteins could play a role in tissue repair and structural remodeling.

ECM1, at approximately 0.01% by weight, is crucial for the structural integrity of the dermis. Its presence in the claimed solution might contribute to the maintenance and enhancement of skin structure and overall health.

Adiponectin, present at about 0.2% by weight, exhibits anti-inflammatory effects and participates in metabolic processes. This protein could potentially support skin health by reducing inflammation and facilitating metabolic functions at the skin level.

Nephroblastoma overexpressed protein, at roughly 0.002% by weight, is implicated in cell adhesion, migration, proliferation, and differentiation. Inclusion of this protein in the claimed solution might support skin healing and regeneration.

Prostacyclin synthase, included at approximately 0.0003% by weight, has a role in promoting vasodilation and inhibiting platelet aggregation. This protein might enhance microcirculation within the skin, improving nutrient and oxygen supply to the skin cells.

C-X-X motif chemokine, present at around 0.001% by weight, is involved in the regulation of immune responses and inflammation and could contribute to skin healing processes.

Heparan sulfate, incorporated at about 2% by weight, participates in a wide variety of biological activities, including cellular growth and differentiation. This component might enhance skin regeneration processes.

Apolipoprotein D, present at approximately 0.01% by weight, is known for its potent antioxidant properties. This could protect skin cells from damage induced by reactive oxygen species or environmental stressors.

The weight percentages assigned to each ingredient reflect the proportion of that ingredient relative to the total weight of the claimed solution.

The ingredients of the claimed solution are described in more detail below. Annexin-1, also known as lipocortin-1, is a protein that is encoded by the ANXA1 gene in humans. This protein is a member of the annexin family, a group of proteins characterized by their ability to bind phospholipids in the presence of calcium ions. In terms of distribution, annexin-1 is found in a wide variety of tissues throughout the body, including but not limited to, the immune cells (like neutrophils, monocytes, and macrophages), epithelial cells, and some types of neurons. It's particularly abundant in cells involved in the inflammatory response.

Functionally, annexin-1 plays several important roles in the body. Its main function is to mediate anti-inflammatory effects, where it works to suppress the actions of cells involved in the body's inflammatory response. It does this by inhibiting the enzyme phospholipase A2, which plays a critical role in the inflammation process by producing arachidonic acid, a precursor to inflammatory mediators like prostaglandins and leukotrienes. Annexin-1 also aids in the process of endocytosis (cellular intake of molecules) and exocytosis (cellular expulsion of molecules), contributes to cell growth and differentiation, and has roles in apoptosis (programmed cell death).

In the pharmaceutical industry, annexin-1's anti-inflammatory properties make it a valuable target for drug development. Drugs that can upregulate or mimic the effects of annexin-1 may potentially be used to treat a variety of inflammatory diseases, such as rheumatoid arthritis, inflammatory bowel disease, and potentially even neuroinflammatory diseases. Moreover, its role in cell growth and apoptosis implies that manipulating annexin-1 activity could also be a therapeutic strategy for cancer treatment.

Galectin-1 is a protein that is encoded by the LGALS1 gene in humans. It is a member of the galectin family, a group of proteins defined by their binding affinity to β-galactoside sugars. Galectin-1 is expressed in a variety of tissues throughout the body. It is particularly abundant in immune cells such as T cells, B cells, macrophages, and dendritic cells, but is also found in many other cell types, including endothelial cells, epithelial cells, and neurons. In terms of function, galectin-1 plays several roles. Its main role involves the regulation of cell-cell and cell-matrix interactions during inflammation, cell proliferation, and apoptosis. It's also involved in modulating the immune response, where it can suppress T-cell activation, induce T-cell apoptosis, and inhibit inflammatory cytokine production, thereby modulating both innate and adaptive immunity.

Galectin-1 has also been found to promote angiogenesis, the process by which new blood vessels form, and this function has been implicated in tumor growth and metastasis. In addition, it has roles in embryogenesis and may be involved in various neurological processes. In pharmaceuticals, galectin-1's involvement in various biological processes makes it an intriguing target for drug development. Its immunomodulatory functions suggest that it could be targeted in autoimmune or inflammatory diseases to decrease aberrant immune responses. Additionally, due to its role in angiogenesis and tumor growth, galectin-1 inhibitors could potentially be utilized in the treatment of certain types of cancer.

S-100 protein is a family of low molecular weight proteins, identified by the fact that they are 100% soluble in saturated ammonium sulfate at neutral pH. They belong to the larger EF-hand superfamily of calcium-binding proteins. The S-100 family is composed of 21 different proteins in humans, with the most well-known being S-100B and S-100A1. The S-100 proteins are expressed in a variety of tissues and cell types throughout the body. The expression of S-100 proteins is cell-type specific. For instance, S-100B is found primarily in glial cells and certain melanocytes, while S-100A1 is largely present in myocardium and skeletal muscle.

In terms of function, S-100 proteins play roles in a variety of intracellular and extracellular functions. Intracellularly, they are involved in the regulation of protein phosphorylation, cytoskeletal components, enzyme activities, cell growth and differentiation, and the inflammatory response. Extracellularly, S-100 proteins might have functions in cell proliferation and differentiation, neuronal survival, and apoptosis. From a pharmaceutical perspective, the varied roles of S-100 proteins make them attractive potential targets for drug development. Elevated levels of S-100 proteins, particularly S-100B, have been found in several neurological conditions such as Alzheimer's disease, schizophrenia, and traumatic brain injury, which suggests that they could be used as biomarkers for these conditions. Additionally, given their role in inflammatory processes, they could potentially be targets for anti-inflammatory drugs.

TIMP-1, or tissue inhibitor of metalloproteinases-1, is a protein encoded by the TIMP1 gene in humans. This protein is a member of the TIMP family, which consists of four proteins (TIMP-1, TIMP-2, TIMP-3, TIMP-4) that are known to regulate the activity of matrix metalloproteinases (MMPs), a group of enzymes involved in the degradation of the extracellular matrix. TIMP-1 is produced in a variety of tissues and cell types throughout the body, including fibroblasts, epithelial cells, and immune cells, among others. It is often found in tissues undergoing remodeling or in response to injury and inflammation.

The primary function of TIMP-1 is to inhibit the activity of MMPs, thereby controlling extracellular matrix degradation. This is essential for maintaining tissue structure and function, but it also has implications in processes such as wound healing, tissue remodeling, and the inflammatory response. Beyond its role as an MMP inhibitor, recent studies have suggested that TIMP-1 may also have other functions, including promoting cell proliferation and survival, and modulating the immune response. In the context of pharmaceuticals, TIMP-1's ability to regulate MMP activity and influence other cellular processes suggests it could be a target for therapeutic intervention. In diseases characterized by excessive tissue remodeling or degradation, such as certain types of cancer, rheumatoid arthritis, and fibrotic diseases, drugs that modulate TIMP-1 activity could potentially be beneficial. Moreover, elevated levels of TIMP-1 have been found in various types of cancer, and it has been suggested that TIMP-1 may contribute to tumor growth, angiogenesis, and resistance to apoptosis. Therefore, inhibitors of TIMP-1 might have potential as anticancer drugs.

TIMP-2, or tissue inhibitor of metalloproteinases-2, is a protein that is encoded by the TIMP2 gene in humans. This protein is a member of the TIMP family, a group of proteins that regulate the activity of matrix metalloproteinases (MMPs), enzymes involved in the degradation of the extracellular matrix. TIMP-2 is expressed in a variety of tissues throughout the body, including connective tissues, skeletal muscle, and the brain, among others. Its production is often upregulated in tissues undergoing remodeling, repair, or in response to inflammation. Like TIMP-1, the primary function of TIMP-2 is to inhibit the activity of MMPs. By controlling the rate of extracellular matrix degradation, TIMP-2 plays a key role in maintaining tissue structure and function. It is involved in various biological processes, including tissue remodeling, wound healing, and the regulation of vascularization. Additionally, TIMP-2 can also influence cell proliferation and apoptosis and modulate the immune response.

In the context of pharmaceuticals, the diverse roles of TIMP-2 suggest it could be a valuable target for therapeutic intervention. Conditions characterized by excessive tissue degradation or remodeling, such as certain cancers, rheumatoid arthritis, and fibrotic diseases, might benefit from drugs that modulate TIMP-2 activity. Moreover, because of its involvement in angiogenesis, TIMP-2 has been investigated for its potential in cancer treatment. It has been suggested that promoting TIMP-2 activity could inhibit tumor growth and metastasis by limiting the development of new blood vessels in tumors.

ECM1, or extracellular matrix protein 1, is a protein that in humans is encoded by the ECM1 gene. As suggested by its name, this protein is a component of the extracellular matrix, which is the non-cellular component present within all tissues and organs that provides not only essential physical scaffolding for the cellular constituents but also initiates crucial biochemical and biomechanical cues required for tissue morphogenesis, differentiation, and homeostasis. ECM1 is expressed in a wide variety of tissues, including the skin, lung, and gastrointestinal tract. It is found in particularly high amounts in areas of active remodeling, such as the basal layer of the epidermis, the placenta, and in various types of tumors.

The functions of ECM1 are diverse and still being elucidated, but it is known to play an important role in maintaining the structure and function of the extracellular matrix. It interacts with several other matrix components, such as perlecan, laminin, and fibulin, thereby influencing tissue integrity. ECM1 is also thought to be involved in processes such as cell adhesion, migration, and angiogenesis. In addition, ECM1 may play a role in inflammatory responses and wound healing. In pharmaceuticals, the potential benefits of targeting ECM1 are primarily being explored in the context of cancer. Given its role in angiogenesis and tissue remodeling, ECM1 could be a potential target for anticancer therapies. ECM1 has been found to be overexpressed in several types of cancer, including breast, ovarian, and esophageal cancers, and has been associated with poor prognosis. Therefore, inhibiting ECM1 could potentially limit tumor growth and spread.

Adiponectin, also known as Acrp30, apM1, GBP28, or AdipoQ, is a protein hormone that is encoded by the ADIPOQ gene in humans. As its name implies, adiponectin is primarily produced by adipose tissue (fat cells), although it is also synthesized to a lesser extent by other tissues like skeletal muscle and the liver. The primary function of adiponectin is to regulate glucose levels and fatty acid breakdown. Specifically, adiponectin enhances the body's sensitivity to insulin, which is the hormone that regulates blood sugar levels. It also promotes fatty acid oxidation, reduces glucose production in the liver, and has anti-inflammatory effects on the lining of the blood vessel walls. Additionally, it plays a role in regulating body weight, where higher levels of adiponectin are typically associated with lower body fat percentages.

In terms of pharmaceutical applications, adiponectin is of particular interest for its potential in managing metabolic diseases like type 2 diabetes and obesity. Given its role in enhancing insulin sensitivity and regulating glucose levels, therapeutics that can increase adiponectin levels or enhance its activity could potentially be used to improve blood sugar control and treat insulin resistance in people with type 2 diabetes. Moreover, since adiponectin levels are inversely correlated with body weight, adiponectin-based treatments could also potentially help in weight management and the treatment of obesity. Adiponectin also has anti-inflammatory and anti-atherogenic properties, suggesting a potential role in treating cardiovascular disease.

Nephroblastoma overexpressed protein, also known as NOV or CCN3, is a member of the CCN family of proteins which also includes cysteine-rich protein 61 (Cyr61/CCN1), connective tissue growth factor (CTGF/CCN2), and Wnt-inducible signaling pathway protein 1 (WISP1/CCN4). NOV is encoded by the NOV gene in humans. This protein is expressed in a variety of tissues throughout the body, including but not limited to the kidney, heart, lung, and vascular system. Its expression is often associated with development and tissue remodeling, and it can also be induced by injury and inflammation. In terms of function, NOV plays a role in a variety of biological processes. It influences cell adhesion, migration, proliferation, and differentiation, and also plays a role in extracellular matrix production. It can influence angiogenesis, the process of new blood vessel formation, and also has roles in inflammation and wound healing. Notably, the protein's name derives from its initial discovery as an overexpressed gene in a Wilms' tumor, a type of pediatric kidney cancer known as nephroblastoma.

In the pharmaceutical context, NOV's diverse functions suggest it may have therapeutic potential. As an important regulator of cell growth and development, NOV could be a target in diseases characterized by abnormal cell growth, such as cancer. Indeed, altered NOV expression has been reported in a variety of cancers, including breast cancer, prostate cancer, and nephroblastoma. However, the role of NOV in cancer is complex, and it may act as either a tumor suppressor or promoter depending on the context. Additionally, given its role in inflammation, angiogenesis, and tissue remodeling, NOV may also be a therapeutic target in inflammatory and fibrotic diseases, as well as in conditions involving abnormal angiogenesis.

Prostacyclin synthase, also known as prostaglandin 12 (prostacyclin) synthase (PTGIS), is an enzyme that in humans is encoded by the PTGIS gene. This enzyme is a member of the cytochrome P450 superfamily of enzymes, which are involved in the synthesis of various molecules in the body. Prostacyclin synthase is found in various tissues throughout the body, including the heart, blood vessels, kidney, and lung. Its presence is particularly prominent in the endothelial cells that line the inner surface of blood vessels. The primary function of prostacyclin synthase is to catalyze the conversion of prostaglandin H2 to prostaglandin 12, also known as prostacyclin. Prostacyclin is a potent vasodilator and inhibitor of platelet aggregation. It helps to prevent blood clots and maintain blood flow, and also has anti-inflammatory effects.

In the context of pharmaceuticals, prostacyclin and its synthase hold considerable interest due to their roles in cardiovascular health. Therapies that can increase the synthesis of prostacyclin, or mimic its actions, may be beneficial for conditions such as hypertension, atherosclerosis, and thrombosis. Prostacyclin analogs have been developed and are used in the treatment of pulmonary arterial hypertension (PAH); a condition characterized by high blood pressure in the arteries leading from the heart to the lungs. These drugs work by mimicking the effects of prostacyclin, helping to relax and widen the blood vessels, and inhibit platelet aggregation. Furthermore, because of prostacyclin's anti-inflammatory effects, there is also interest in its potential for treating other inflammatory diseases.

C-X-X motif chemokine refers to a broad class of chemokines, a type of cytokine, which are characterized by the presence of two cysteine residues separated by any two other amino acids, represented as “X.” The C-X-X motif, or CXC motif, is an important structural characteristic that influences the function of these proteins. Examples of proteins in this group include CXCL8 (also known as Interleukin-8), CXCL12 (Stromal cell-derived factor-1), and many others.

Chemokines are small cytokines or signaling proteins secreted by cells. They have the ability to induce directed chemotaxis in nearby responsive cells, hence the name “chemotactic cytokines.” They are produced by a variety of cells including immune cells, fibroblasts, and endothelial cells among others, often in response to an immune stimulus or during development. CXC chemokines primarily attract neutrophils to the site of infection to combat pathogens and are also involved in the angiogenesis process. They are also involved in various other biological processes such as cell migration, immune responses, hematopoiesis, and organogenesis.

The CXC chemokines play a crucial role in the body's immune response, and as such, they are of significant interest in pharmaceutical research and development. They can serve as potential targets for therapeutic intervention in a variety of diseases that involve inflammation and immune responses. For example, inhibiting the activity of certain CXC chemokines could potentially be beneficial in treating inflammatory diseases like rheumatoid arthritis or psoriasis. In the context of cancer, certain CXC chemokines are known to influence tumor growth, angiogenesis, and metastasis, making them potential targets for anticancer therapies.

Heparan sulfate is a linear polysaccharide found on the cell surface and in the extracellular matrix of a wide range of tissues. It's a member of the glycosaminoglycan family, which also includes substances like heparin, chondroitin sulfate, and hyaluronic acid. Heparan sulfate is ubiquitously found on the surface of cells in virtually all animal tissues. It is primarily located on the cell surface or in the extracellular matrix, where it's usually attached to proteins to form proteoglycans. These heparan sulfate proteoglycans are integral to many biological processes. Functionally, heparan sulfate plays a significant role in a variety of biological activities. These include cellular adhesion and migration, organization of the extracellular matrix, regulation of cell growth and differentiation, and involvement in various signaling pathways. Notably, it's also involved in blood coagulation and has anticoagulant properties.

In the pharmaceutical industry, heparan sulfate and its closely related analogue, heparin, have been exploited for their anticoagulant properties. Heparin is widely used as an injectable anticoagulant and has the highest negative charge density of any known biological molecule. It's used in the treatment and prevention of thrombosis, or blood clots, as well as certain medical procedures that carry a high risk of clot formation. Additionally, because heparan sulfate is involved in many biological processes, it could potentially be targeted for other therapeutic purposes. For instance, its role in cellular growth and differentiation could potentially be harnessed in regenerative medicine, and its role in cell adhesion and migration could be targeted in the treatment of metastatic cancer.

Apolipoprotein D (ApoD), encoded by the APOD gene in humans, is a member of the lipocalin protein family, known for their role in transporting small hydrophobic molecules. ApoD is a glycoprotein that is part of the high-density lipoprotein (HDL) complex, often referred to as “good cholesterol.” ApoD is expressed in a variety of tissues throughout the body, including the brain, adrenal glands, kidneys, liver, and several others. It's also present in various bodily fluids like plasma, cerebrospinal fluid, and breast milk. The exact functions of ApoD are still not fully understood, but research indicates it plays a role in several biological processes. It's involved in lipid transport and metabolism, and also appears to have antioxidant properties. Moreover, ApoD may play a role in the regulation of inflammation and response to stress, particularly in the nervous system.

From a pharmaceutical standpoint, the study of ApoD is particularly interesting due to its implication in several pathological conditions. Elevated levels of ApoD have been observed in several diseases, including neurodegenerative disorders like Alzheimer's and Parkinson's disease, various types of cancer, and in response to brain injury. It is thought that the increased ApoD levels might be a protective response, as its antioxidant and anti-inflammatory properties could help mitigate damage in these conditions. There is ongoing research into whether ApoD could be targeted therapeutically to treat or prevent such diseases. It's hypothesized that enhancing ApoD activity might have neuroprotective effects, for instance, slowing the progression of neurodegenerative diseases. In addition, given its role in lipid metabolism, ApoD could potentially be targeted in the treatment of dyslipidemia and related metabolic disorders.

Given the wide range of proteins included in the claimed solution and their diverse biological functions, the formulation may be used in a variety of contexts related to skin health and disease. The claimed solution may be used in would healing and repair. Proteins like annexin-1, galectin-1, the S-100 proteins, TIMP-1, TIMP-2, nephroblastoma overexpressed protein, and ECM1 have roles in cellular adhesion, migration, proliferation, and differentiation, which are all critical processes in wound healing and skin repair. Heparan sulfate could also aid in these processes due to its role in cell growth and differentiation.

The claimed solution may be used in treating inflammatory skin conditions. Proteins like annexin-1, adiponectin, galectin-1, and the C-X-X motif chemokine have anti-inflammatory properties. Therefore, the claimed solution may be used to soothe inflammatory skin conditions such as eczema, psoriasis, or dermatitis. The claimed solution may also be used in treating skin aging and wrinkles. The presence of TIMP-1 and TIMP-2, which are inhibitors of enzymes that break down the extracellular matrix, suggests that the claimed solution could potentially be used in the context of skin aging. By inhibiting the breakdown of the extracellular matrix, these proteins help maintain skin firmness and potentially reduce the appearance of wrinkles. The claimed solution may further be used in skin hydration and barrier function. ECM1 plays a role in the structural integrity of the dermis and maintaining skin barrier function, which could imply a potential use of the claimed solution in improving skin hydration and barrier function. Additionally, the claimed solution may be used in treating oxidative stress. Given the antioxidant properties of apolipoprotein D, the claimed solution may be used to protect the skin from oxidative stress, which could be beneficial in the context of environmental damage or aging.

Also, the claimed solution may be used in microcirculation. Prostacyclin synthase promotes vasodilation, which helps improve microcirculation in the skin, improving nutrient and oxygen supply to skin cells. Finally, the claimed solution may be used in cancer therapy support. Given that some of the proteins, such as galectin-1, have been associated with cancer progression, the solution may be used in conjunction with cancer therapies.

The claimed solution is distinctive from other known topical solutions due to its unique combination of specific proteins at carefully considered concentrations. Many of these proteins are involved in complex biological processes relevant to skin health, wound healing, inflammation, and cellular integrity, and it is uncommon to find all of these components combined in a single topical formulation.

Most commercially available topical solutions focus on simpler compounds like moisturizers (which include elements such as hyaluronic acid or ceramides), retinoids (vitamin A derivatives), or other specific active ingredients like salicylic acid for acne or corticosteroids for inflammatory skin conditions. These solutions often have one or a few active ingredients that serve a specific purpose, such as moisturizing the skin, reducing inflammation, or promoting cellular turnover.

In contrast, the claimed solution contains a broad array of proteins, each with distinct and potentially synergistic roles. This allows for a more comprehensive approach to skin health, addressing multiple aspects of skin biology simultaneously. For example, some proteins in the claimed solution promote wound healing and skin repair (e.g., annexin-1, TIMPs), while others reduce inflammation (e.g., adiponectin, galectin-1) or enhance skin's structural integrity (e.g., ECM1).

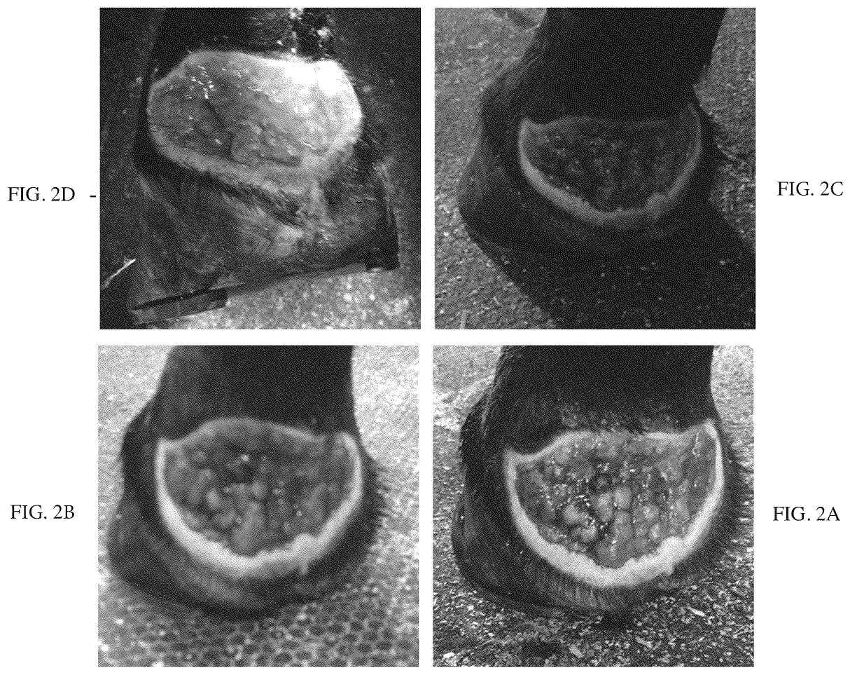

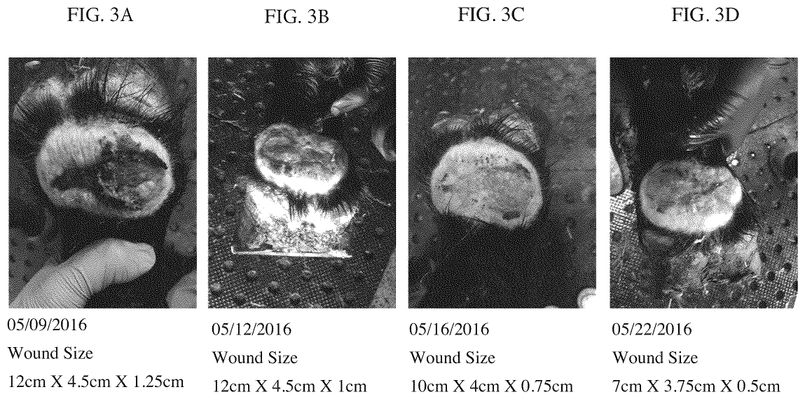

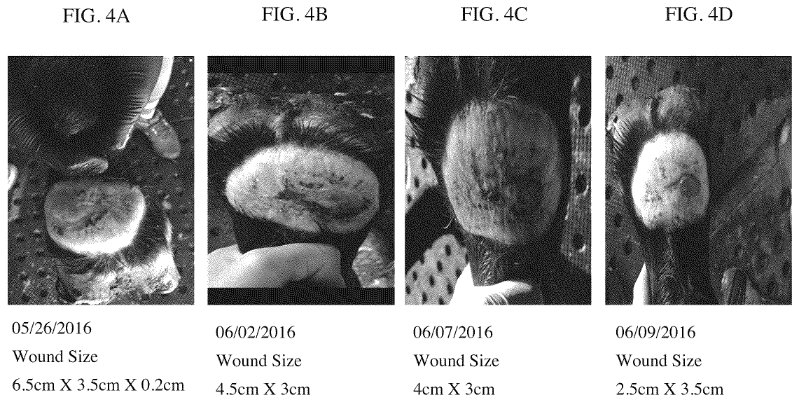

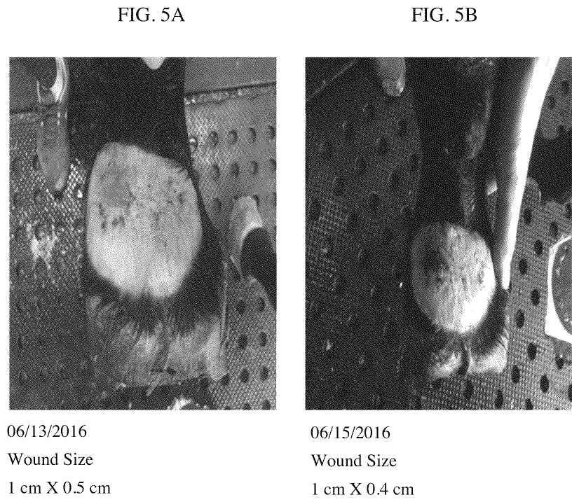

In a first example of experimental results, a three-year-old female horse suffered a severe burn during the removal of a wart on the back of the right front foot. The wound persisted for 2 months without any substantial healing. The picture shown in FIG. 2A was taken the first week after the burn occurred. Treatment with the UCT Wharton's jelly extract (i.e., the product produced by the claimed subject matter) began on May 9, 2016, with the extract being applied directly to the wound 3 times a week. The wound was wrapped, and measurements were taken. FIGS. 2A, 2B, 2C, and 2D show the progression of a wound over time, with FIG. 2A being the photograph taken earliest in time and FIG. 2D being the photograph taken latest in time. FIGS. 3A, 3B, 3C, 3D, 4A, 4B, 4C, 4D, 5A, and 5B show photographs of the same wound (shown in FIGS. 2A-2D) at various dates indicated, as well as the size of the wound, which clearly shows that the wound heals over time, due to the use of the product produced by the claimed subject matter. FIG. 3A, for example, shows a 5-9-2016 photograph of a wound sized 12 cm×4.5 cm×1.25 cm, while the FIG. 3D shows a 5-22-2016 photograph of a wound sized 7 cm×3.75 cm×0.5 cm.

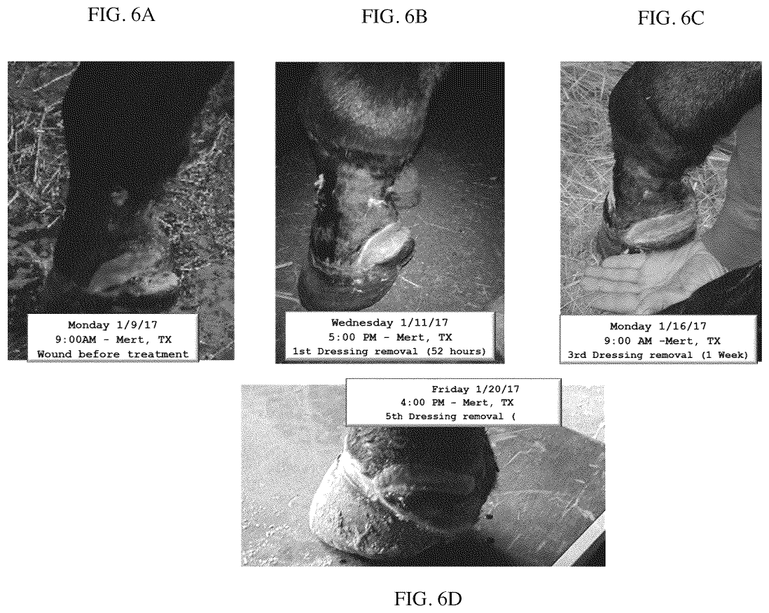

In a second example of experimental results, a horse that suffered a severe laceration to its hoof area. The product produced by the claimed subject matter was combined in a moisturizer like cream and applied to the wound 2× daily. FIGS. 6A, 6B, 6C, and 6D show photographs of the wound at various dates indicated, as well as the size of the wound, which clearly shows that the wound heals over time. FIG. 6A, for example, shows a 9 am 1-9-17 photograph taken in Mert, TX of a wound before treatment with the product produced by the claimed subject matter, while FIG. 6D shows a 4 pm 1-20-17 photograph taken in Mert, TX of said wound after treatment with the product produced by the claimed subject matter, clearly showing improvement.

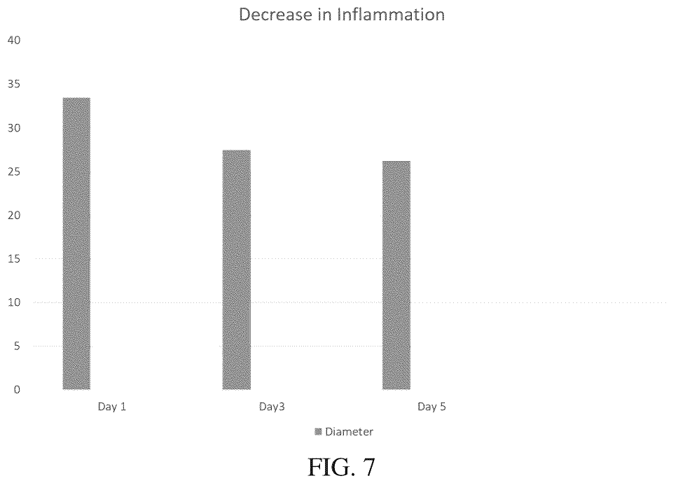

In a third example of experimental results, a 7-year-old male horse that broke his left fetlock at age 2. He has suffered from chronic suspensory branch lesions with scar tissue formation. The horse has been bandaged constantly and has had corrective shoeing performed to improve soundness with no improvements. The product produced by the claimed subject matter was applied to this horse 2× a day for 7 days with the following results demonstrating the decrease in inflammation in a very short period. FIG. 7 shows the decrease in inflammation due to the wound exhibited by the horse over time. FIG. 7 shows that the wound was about 32 cm in diameter on day 1 but shrank to about 26 cm in diameter at day 3 of the treatment.

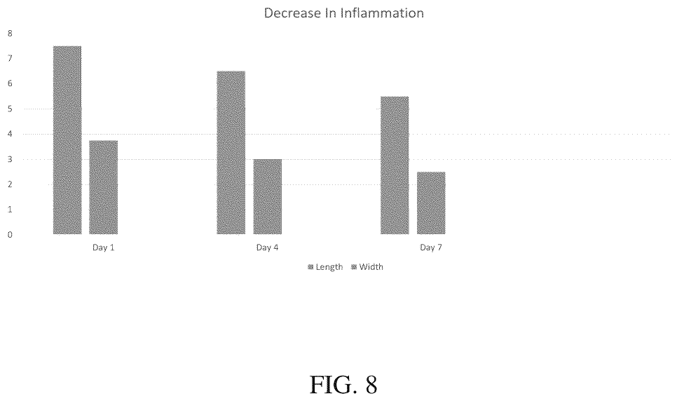

In a fourth example of experimental results, a six-year-old male horse that suffered an injury where the splint bones popped. The injury did not heal resulting in physical calcification of the splint bone to the cannon bone causing inflammation and sensitivity to the suspensory ligament. The product produced by the claimed subject matter was applied 2× a day for 7 days. The injury occurred 4 months ago and was initially treated with surpass and poultice clay with no effect. This treatment was performed 4 months before the application of the extract. FIG. 8 shows the decrease in inflammation due to the wound exhibited by the horse over time. FIG. 8 shows that the wound was about 7.5 cm in length on day 1 but shrank to about 5.5 cm in length at day 7 of the treatment.

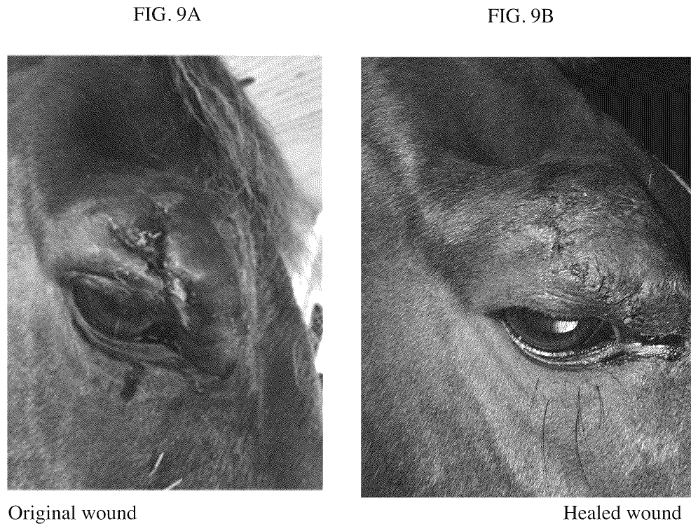

In a fifth example of experimental results, horse suffered a laceration above its eye that required sutures. The product produced by the claimed subject matter was administered after the sutures were performed 2 times a day. The results of wound closure were from 3 days application of the claimed product. FIG. 9A shows the original wound, while FIG. 9B shows the fully healed wound.

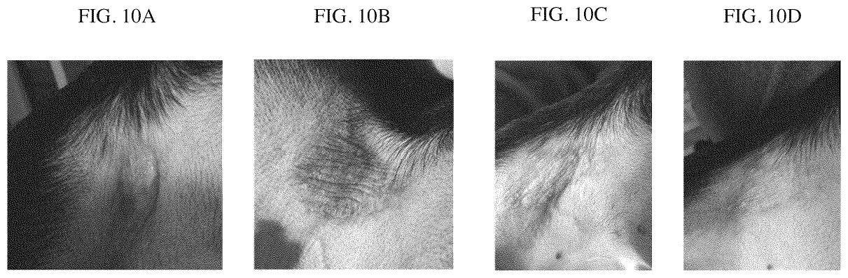

In a sixth example of experimental results, an 8-month-old puppy exhibited an ulcer on its pectoral area that turned out to be a Staph aureus infection (confirmed by independent testing lab and an independent physician). After four days of treatment with the product produced by the claimed subject matter and a wound cream, there was total clearance of the Staph infection. FIGS. 10A, 10B, 10C, and 10D show photographs of the wound at different dates, which clearly shows that the wound heals over time. FIG. 10A is the photograph taken earliest in time and FIG. 10D being the photograph taken latest in time. Anti-microbial activity could be attributed to 2 possible mechanisms: the first is that the umbilical extract contains antimicrobial peptides that play a direct role in the antimicrobial activity, or secondly, there are numerous cytokines and immune signaling molecules present in the claimed produce that there is a massive influx of immune cells into the location where the cream has been administered.

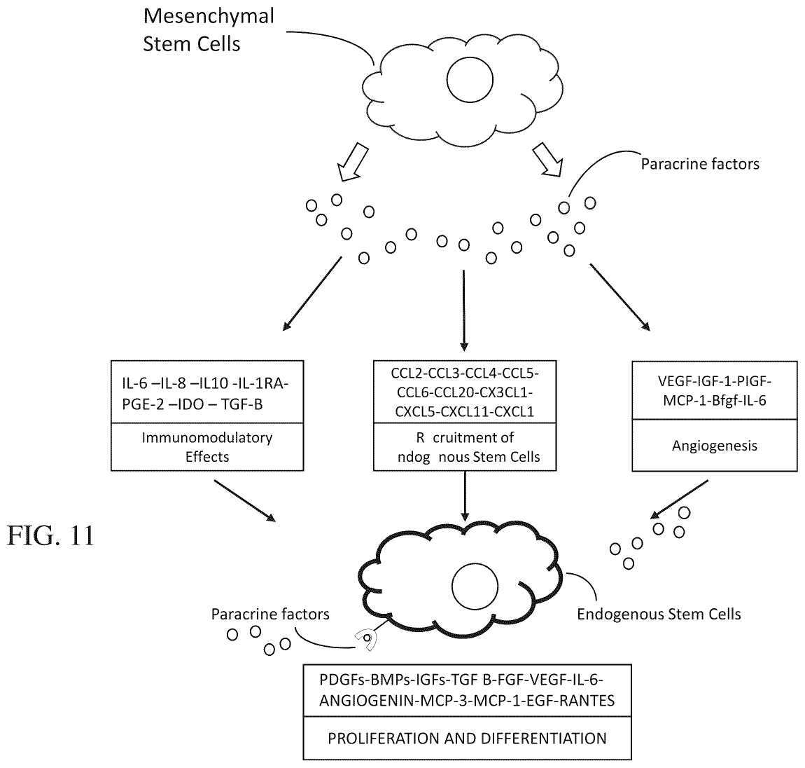

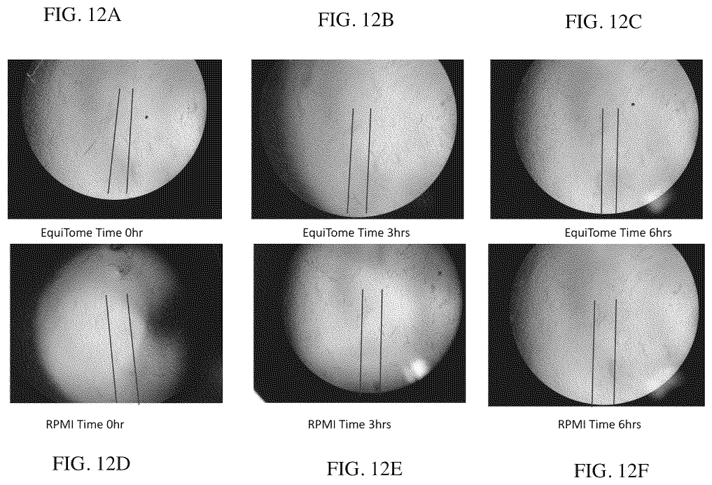

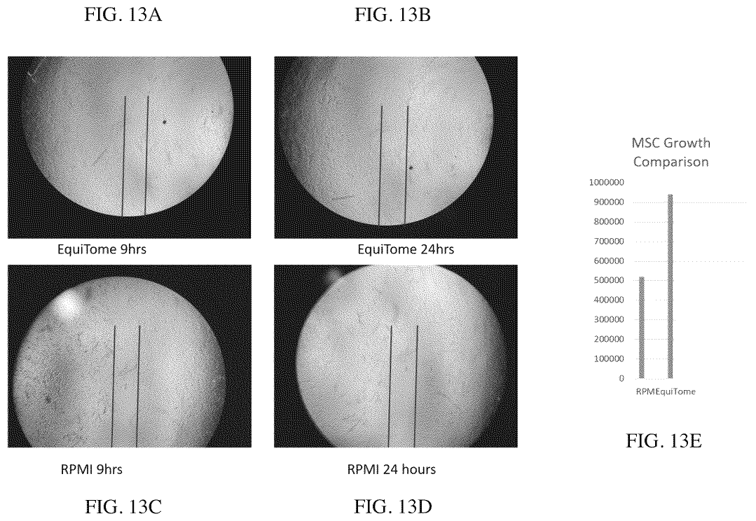

One possible mechanism of action of the product produced by the claimed subject matter is that it recruits endogenous stem cells to the location that it is applied to or injected into. See FIG. 11 for a description of said mechanism, which includes the paracrine factors, and shows how the immunomodulatory effects, angiogenesis and recruitment of endogenous stem cells are processed for proliferation and differentiation. In an attempt to demonstrate this experimentally, MSC migration assays were performed. MSCs were seeded into culture containing a filter paper soaked in product produced by the claimed subject matter and a control was performed with the filter paper soaked in standard RPMI culture media. The cells in the product produced by the claimed subject matter media can be clearly seen migrating and filling in the area where the filter paper was at the 24-hour time point (paper removed to show the cells) and with the control media, RPMI, no cell migration can be seen. See FIGS. 12A-13E. FIG. 12A shows culture media with the claimed subject matter at an initial time, while FIG. 12B shows the media after 3 hours, FIG. 12C shows the media after 6 hours, FIG. 13A shows the media after 9 hours, and FIG. 13B shows the media after 24 hours. FIG. 12D shows the control RPMI culture media at an initial time, while FIG. 12E shows the RPMI culture after 3 hours, FIG. 12F shows the RPMI culture after 6 hours, FIG. 13C shows the RPMI culture after 9 hours, and picture FIG. 13D shows the RPMI culture after 24 hours. FIG. 13E shows an MSC growth comparison graph. Additionally, it was observed that MSCs proliferation was always greater in media supplemented with the product produced by the claimed subject matter versus media supplemented with FBS. Thus, the product produced by the claimed subject matter can be used as cell culture.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A solution for topical application to the skin, comprising of:
about 0.05% by weight of annexin-1;
about 0.76% by weight of galectin-1;
about 0.23% by weight of protein S-100;
about 0.003% by weight of timp-1;
about 0.01% by weight of timp-2;
about 0.01% by weight of ECM1;
about 0.2% by weight of adiponectin;
about 0.002% by weight of nephroblastoma overexpressed protein;
about 0.0003% by weight of prostacyclin synthase;
about 0.001% by weight of C-X-X motif chemokine;
about 2% by weight of heparan sulphate; and
about 0.01% by weight of apolipoprotein D.
